(19) United States
(12) Patent Application Publication
(10) Pub. No.: US 2018/0274384 A1
(43) Pub. Date: Sep. 27, 2018

(54) SENSOR AND HOUSING DESIGN FOR IN-LINE FLUID MONITORING

(71) Applicant: ExxonMobil Research and Engineering Company, Annandale, NJ (US)

(72) Inventors: Christopher Vander Neut, Spring, TX (US); Steven M. Jetter, Hightstown, NJ (US); Michael L. Alessi, Rose Valley, PA (US)

(21) Appl. No.: 15/916,612

(22) Filed: Mar. 9, 2018

Related U.S. Application Data
(60) Provisional application No. 62/474,664, filed on Mar. 22, 2017.

(51) Int. Cl. F01D 17/08; G01N 21/25; G01N 21/64; G01N 21/85; G01N 33/28; G01N 21/88; F01D 17/26

(52) U.S. Cl. CPC F01D 17/08; G01N 21/25; G01N 21/64; G01N 21/85; G01N 2201/06193; G01N 21/8806; F01D 17/26; G01N 2021/6439; G01N 33/28

(57) ABSTRACT

A working fluid monitoring system for monitoring a working fluid of a working fluid system of a piece of equipment. The working fluid monitoring system can include a housing member having a working fluid passage for receiving the working fluid therein and a sensor in operable communication with the working fluid within the working fluid passage of the housing member configured to monitor in situ the working fluid and output a sensor signal. The working fluid monitoring system can be quickly and conveniently retrofitted to existing machinery for in situ monitoring, detecting, testing, and/or validating of a working fluid, such as oil, hydraulic fluid, and the like.

### CROSS-REFERENCE TO RELATED APPLICATIONS

This application claims the benefit of U.S. Provisional Application No. 62/474,664, filed on Mar. 22, 2017, the entire contents of which are incorporated herein by reference.

### FIELD

The present disclosure relates to working fluid monitoring and, more particularly, relates to a sensor and housing design for in-line working fluid monitoring.

### BACKGROUND

This section provides background information related to the present disclosure which is not necessarily prior art.

Machinery of all kinds is used throughout the world for advanced applications and uses. Such machinery often uses a working fluid, such as oil, hydraulic fluid, or the like, for functional purposes, such as lubrication, actuation, and manipulation of an implement. However, many of these applications are in remote areas of the world that make it difficult to repair and maintain such machinery. Consequently, it is important to ensure that such machinery is properly operated and maintained to minimize the potential for downtime due to repair and maintenance.

In some situations, it has been found that maintenance personnel may attempt to service, repair, or maintain such machinery with improper fluids, counterfeit fluids, and/or incompatible fluids. These fluids can have a negative effect on the reliability and usefulness of the machinery, and can negatively impact the perceived overall reliability of the machinery from a particular manufacturer. Therefore, as can be appreciated by those skilled in the art, it is often important to monitor parameters of the working fluid to insure the fluid is appropriate for the application, dependable, and of initial and continued serviceability quality.

Attempts have been made to provide monitoring of such working fluid. These conventional attempts typically include using a sensor that is screw-mounted into systems of the machinery via a customized threaded access port that permits the sensor or probe to be positioned in the flow path of the working fluid. Unfortunately, this type of threaded access port configuration is often used to retrofit existing machinery in the field and thus requires the machinery to be permanently modified to include the customized threaded access port. Consequently, there is a high likelihood of damage to the machinery due to improper port location, introduction of foreign contaminants into the system from the drilling process, and leakage due to improper sealing in the potentially-pressurized system. It should be understood that in any conventional monitoring system that may require additional components besides a sensor (e.g. light source, flow meter, etc.), the need for additional threaded access ports is increased and results in increased likelihood of damage, contaminants, leakage, and the like.

Accordingly, there exists a need in the relevant art to provide a system for permitting the monitoring of a working fluid of machinery. Furthermore, there exists a need in the relevant art to provide a retrofitable system for monitoring the working fluid of the machinery that comprises a simple and reliable installation without the need to permanently modify the machinery. Still further, there exists a need in the relevant art to provide a system for monitoring the working fluid of the machinery that overcomes the disadvantages of the prior art.

### SUMMARY

This section provides a general summary of the disclosure, and is not a comprehensive disclosure of its full scope or all of its features.

According to the principles of the present teachings, in some embodiments, a working fluid monitoring system for monitoring a working fluid of a working fluid system of a piece of equipment is provided having advantageous construction and method of use. The working fluid monitoring system can include a housing member having a working fluid passage for receiving the working fluid therein and a sensor in operable communication with the working fluid within the working fluid passage of the housing member configured to monitor in situ the working fluid and output a sensor signal. The working fluid monitoring system can be quickly and conveniently retrofitted to existing machinery for in situ monitoring, detecting, testing, and/or validating of a working fluid, such as oil, hydraulic fluid, and the like.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a machinery having a working fluid monitoring system according to the principles of the present teachings;

FIG. 2 is a perspective view of a working fluid monitoring system for use in a tubular member;

FIG. 3 is a perspective view of a working fluid monitoring system having a mesh screen section for use in a tubular member;

FIG. 4 is a perspective view of a working fluid monitoring system mountable to a flanged end of a working fluid fill pipe;

FIG. 5 is a perspective view of a working fluid monitoring system having internal baffles for use in a tubular member;

FIG. 6 is a perspective view of a working fluid monitoring system having collared ends for use in an inline tubular member; and

FIG. 7 is a cross-sectional view of a working fluid monitoring system having a threaded male end for threaded coupling with an existing working fluid fill port and an opposing threaded female end.

Corresponding reference numerals indicate corresponding parts throughout the several views of the drawings.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

According to the present teachings, a working fluid monitoring system for monitoring a working fluid of a working fluid system of a piece of equipment is provided. The working fluid monitoring system can include a housing member having a working fluid passage for receiving the working fluid therein and a sensor in operable communication with the working fluid within the working fluid passage of the housing member configured to monitor in situ the working fluid and output a sensor signal.

The working fluid monitory system promotes quick and convenient installation and initiation of a sensor into any machinery or equipment. With the increase need for sensors, especially in connection with the use of fluid-based markers, in recent years, provides a solution particularly adapted for testing, development, and commercial monitoring. The housing member is configured to permit non-destructive installation for faster installation and quicker return to service for the machinery. The sensor design enables fresh working fluid sensing and measurement for fluid identification or other purposes, and includes several design and installation options to accommodate various machine configurations.

In particular, as will be discussed herein, the working fluid monitoring system can be configured such that the housing member includes a fill pipe of the piece of equipment, wherein the fill pipe is configured to receive the working fluid there through, such as during fluid replacement. The housing member can be a hollow cylindrical member that is disposed or positioned within the fill pipe of the piece of equipment. In some applications, the housing member can engage the fill pipe of the piece of equipment to maintain a predetermined position within the fill pipe. Alternatively, the housing member can be mounted within a fill port of the piece of equipment, which is typically used for filling of replacement working fluid. The housing member can include a flanged fitting on an end to permit it being mounted to an existing fill pipe flange connection. In some applications, the housing member can include collared fittings on opposing ends thereof for mounting within an existing pipe of the piece of equipment.

As will be further discussed herein, the working fluid monitoring system can include a light source configured to output light within the housing member and upon the working fluid, wherein the sensor is responsive to the working fluid when exposed to the light. The light source can be an ultraviolet light source. Similarly, the sensor can be an RGB light sensor, a sensor that detects a fluorescence of the working fluid within the housing member, a sensor is configured to monitor in situ a parameter of the working fluid, and/or a sensor configured to detect a counterfeit working fluid. It should also be understood that the sensor can be any type of sensor, probe, cable, or other electronic means for working fluid monitoring and/or detection. The sensor can be configured to monitor in situ a parameter of the working fluid chosen from the group consisting of suspended soot, dielectric, conductivity, capacitance, impedance, light transmission, acoustic resonance, vapor pressure, pressure, temperature, total acid number, total base number, pH, oxidation, viscosity, flow rate, flow volume, water, coolant, density, oil viscosity grade, VI, SSI (viscosity modifier shear stability), ferrous iron, fuel dilution, RGB color, florescence, phosphorescence, UV, infrared, X-ray, XRF, RF, refractive index, varnish, deposits, iron, lead, copper, aluminum, tin, vanadium, zinc, phosphorus, boron, calcium, magnesium, include zirconium, nitrogen, chlorine, silver, titanium, chromium, cobalt, nickel, molybdenum, yttrium, tungsten, gold, cerium, strontium, sulfur, particle count, and air concentration.

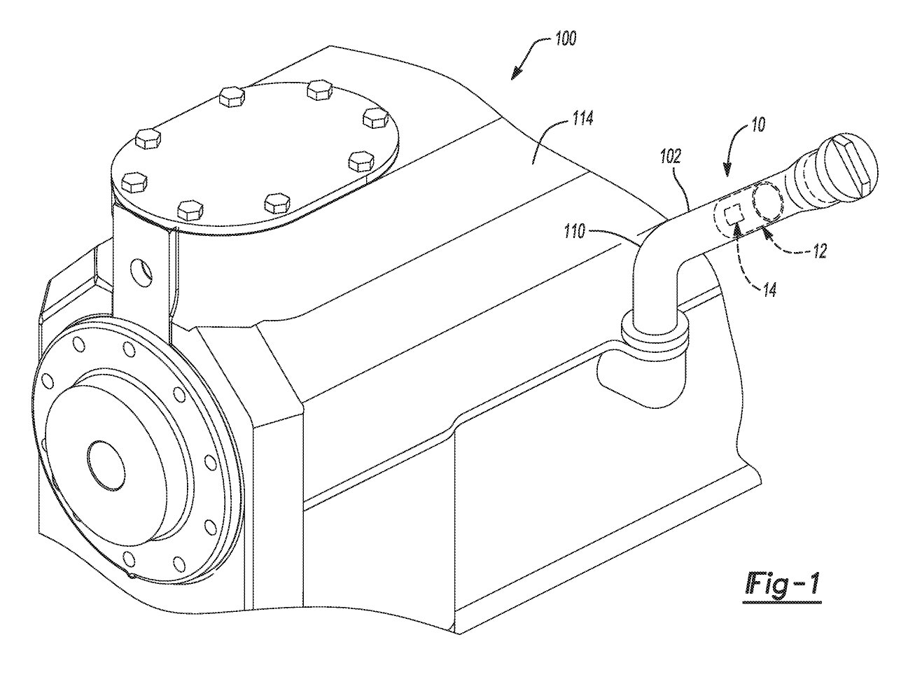

With particular reference to the figures, the working fluid monitoring system of the present teachings will be described in greater detail. As illustrated in FIGS. 1, 4, 6, and 7, working fluid monitoring system 10 can be used in connection with machinery 100, such as an engine, compressor, pump, actuator, working implement, drive system, articulation system, or other device having a working fluid to be monitored. In some exemplary applications, machinery 100 can comprise an engine having a lubrication system, wherein the oil within the lubrication system is termed the working fluid. However, it should be understood that within the context of the present disclosure, unless specifically stated otherwise, the term working fluid includes lubrication fluid, hydraulic fluid, gear oil, transmission oil, grease, fuel, coolant, refrigeration fluid, brake fluid, or other fluids used within the machinery. Moreover, it should be understood that although the present teachings will be described in connection with machinery 100 being an engine in the interest of brevity, the present application should not be regarded as being limited to only applications and uses requiring an engine. By way of non-limiting example, the present teaching may provide utility in a wide variety of applications, including marine, agricultural, industrial, or any application benefiting from fluid monitoring.

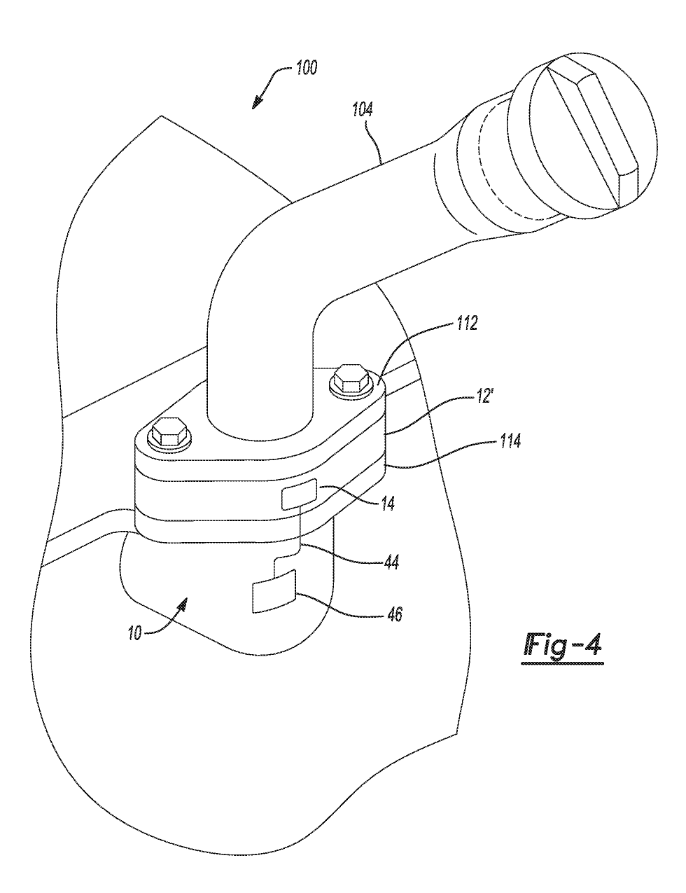

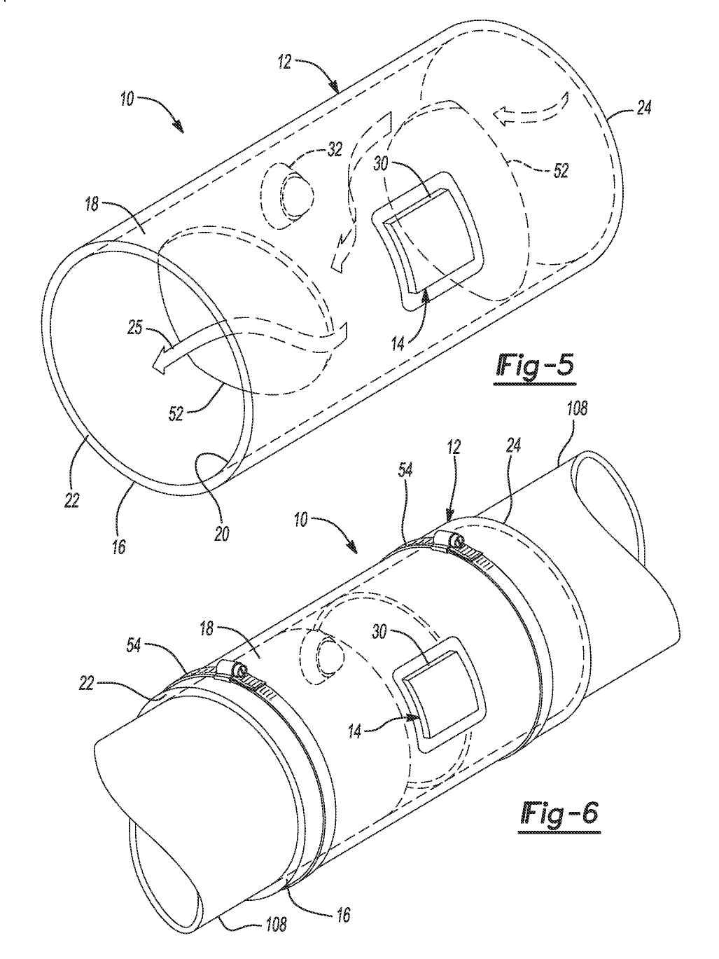

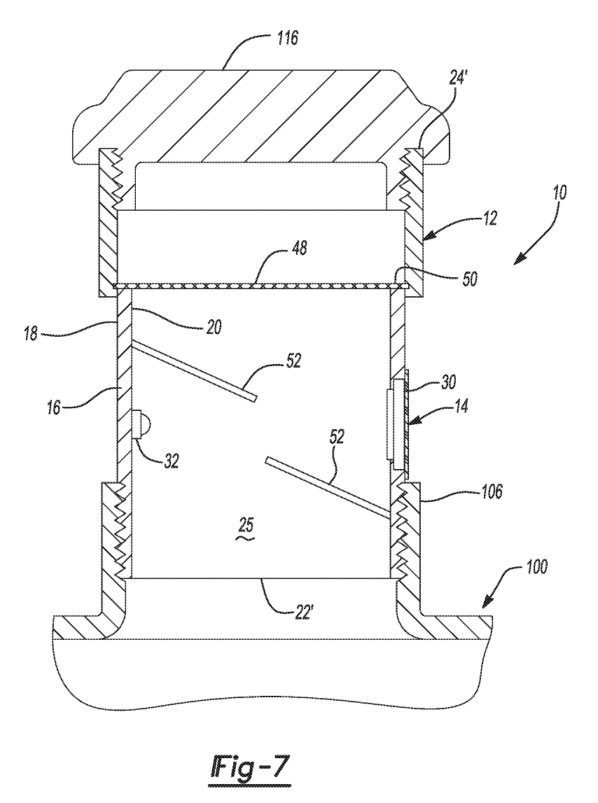

As illustrated in the several figures, machinery 100 can comprise a fill pipe 102 (FIG. 1), a flanged fill pipe 104 (FIG. 4), a threaded fill port 106 (FIG. 7), and/or tubing 108 (FIG. 6). These features may be part of or incorporated into a conventional lubrication system of the engine, and may include a sump, oil pump, oil lines, oil spurt holes and galleries, pressure relief valves, and an oil filter. The lubrication system can be configured to pump, filter, cool, and distribute oil throughout the engine.

As illustrated in FIGS. 1-3, in some applications working fluid monitoring system 10 can be positioned within a fill pipe 102 of machinery 100 for monitoring the working fluid during a fill process, whereby working fluid, such as lubrication oil, is added to the sump via the fill pipe 102. During this operation, the lubrication oil passes through fill pipe 102 to enter the lubrication system of the engine. As will be appreciated, working fluid monitoring system 10 is configured to permit the working fluid (e.g. lubrication oil) to flow there through or there past and be exposed to a sensor.

With continued reference to FIGS. 1-3, in some embodiments, working fluid monitoring system 10 can comprise a housing member 12 having a sensor system 14 coupled, integrated, disposed, and/or affixed thereto. Although it should be appreciated that the shape of housing member 12 can vary depending upon the associated structure of machinery 100 and/or its application or use, for purposes of the present disclosure housing member 12 will be described in connection with conventional fill pipe 102 having a cylindrical cross-section. Accordingly, in some embodiments, housing member 12 can be a generally-tubular or hollow member defining a continuous sidewall 16 having an outer surface 18, an inner surface 20, and opposing ends 22, 24. Housing member 12 can further include a working fluid passage 25 extending therethrough to permit passage of working fluid therethrough.

According to the principles of the present teachings, working fluid monitoring system 10 can comprise any one or a number of sensor systems 14. Sensor system 14 can be disposed in any orientation to permit the monitoring of the working fluid. For example, sensor system 14 can be disposed within the flow of the working fluid and/or disposed in fluid communication with the working fluid. It should be noted that sensor system 14 can be coupled to provide direct communication or contact with the working fluid (e.g. within the flow of the working fluid or within a port having direction communication with the working fluid). Additionally, sensor system 14 can be coupled to provide indirect communication with the working fluid (e.g. separate from direct communication, but within operable range for measurement purposes).

Likewise, housing member 12 can be made of any material suitable for the application and use environment, such as being configured to accommodate environmental heat, pressure, contamination, flowrate, vibration, harshness, and the like. In some applications, housing member 12 can be made of a clear, transparent, semi-transparent, translucent, or opaque. Such material selection can permit sensor system 14 to detect a parameter of the working fluid through housing member 12, thereby permitting sensor system 14 to be mounted within sidewall 16 (e.g. at least partially encased) or upon outer surface 18 of housing member 12 (e.g. via epoxy or other fastening or adhesive system). In this regard, a continuous inner surface 20 can be maintained over sensor system 14, thereby ensuring sensor system 14 is not directly exposed to the working fluid.

Sensor system 14 can be used to measure, monitor, detect, test, and/or validate the working fluid. This is particular advantageous for monitoring any one or a number of parameters of the working fluid, such as, but not limited to, the suspended soot, dielectric, conductivity, capacitance, impedance, light transmission, acoustic resonance, vapor pressure, pressure, temperature, total acid number, total base number, pH, oxidation, viscosity, flow rate, flow volume, water, coolant, density, oil viscosity grade, VI, SSI (viscosity modifier shear stability), ferrous iron, fuel dilution, RGB color, florescence, phosphorescence, UV, infrared, X-ray, XRF, RF, refractive index, varnish, deposits, iron, lead, copper, aluminum, tin, vanadium, zinc, phosphorus, boron, calcium, magnesium, include zirconium, nitrogen, chlorine, silver, titanium, chromium, cobalt, nickel, molybdenum, yttrium, tungsten, gold, cerium, strontium, sulfur, particle count, air concentration, and the like. Sensor system 14 can further be used to measure or monitor associated and/or nearby systems (e.g. working fluid system) that may result in a change in working fluid parameters, such as a coolant leak, faulty or failing oil pump, or the like.

Sensor system 14 can further be used to detect counterfeit working fluid that is not suitable for use in machinery 100. That is, it is known that some machinery operators and/or maintenance personnel may knowingly or unknowingly attempt to add working fluid to machinery 100 that is not particularly designed or approved for such machinery or application. This can even occur when working fluid has been packaged to appear to be from a source from which it is not—specifically, it has been found that in some remote locations, unscrupulous sellers will attempt to pass off lubrication oil or hydraulic fluid as being from an approved manufacturer by simply adding reused and/or inferior fluid to a previously-used container. This reused and/or inferior fluid however does not contain the same fluid-based additive markers and, thus, can be easily detected by sensor system 14. Upon detection of such fluid, sensor system 14 can output a sensor signal 26 alerting the owner, operator, maintenance personnel; the control system or central processing unit of machinery 100; and/or the manufacturer of machinery 100 (collectively referenced as numeral 28).

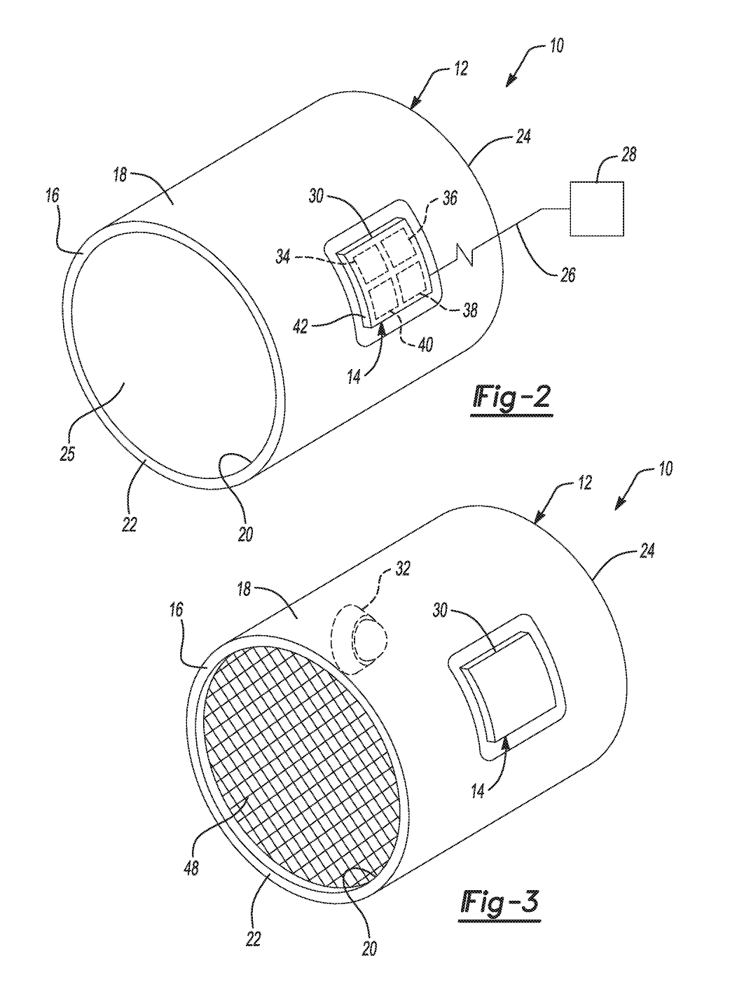

In some embodiments, sensor system 14 can comprise any combination of sensors, probes, flowmeters (collectively referred to as sensor component 30) and optional light sources 32. Sensor component 30 can be operably coupled to housing member 12 to be reliably retained within a position relative to housing member 12. Accordingly, as briefly described herein, sensor component 30 can be affixed via epoxy, adhesive, or other fastening system to outer surface 18, inner surface 20, or completely or partially encapsulated within sidewall 16 of housing member 12. The particular mounting structure of sensor component 30 to housing member 12 will be dependent upon the parameter to be monitored in the working fluid, the environment in which the working fluid monitoring system 10 is disposed, and other operational considerations. In some embodiments, as illustrated in FIGS. 2, 3, 5, and 6, sensor component 30 can be mounted or affixed to outer surface 18 of housing member 12. In this regard, housing member 12 is made of a material that permits monitoring of the working fluid through sidewall 16 of housing member 12 and, therefore, sensor component 30 is in indirect communication with the working fluid. In some embodiments, as illustrated in FIG. 7, sensor component 30 can be mounted or affixed to inner surface 20 of housing member 12. In the regard, sensor component 30 is in direct communication with the working fluid and, therefore, sensor component 30 should be selected to withstand any associated operating parameters.

Similarly, depending upon the sensor component 30 being used, an optional light source 32 can be operably coupled to housing member 12 to be reliably retained within a position relative to housing member 12 and in operational proximity to sensor component 30. Accordingly, as briefly described herein, light source 32 can be affixed via epoxy, adhesive, or other fastening system to outer surface 18, inner surface 20, or completely or partially encapsulated within sidewall 16 of housing member 12. The particular mounting structure of light source 32 to housing member 12 will be dependent upon the parameter to be monitored in the working fluid, the environment in which the working fluid monitoring system 10 is disposed, and other operational considerations. In some embodiments, as illustrated in FIGS. 3, 5, 6, and 7, light source 32 can be mounted or affixed to inner surface 20 of housing member 12. In the regard, light source 32 is in direct communication with the working fluid and oriented in such a way as to complement reliable operation of sensor component 30. Light source 32 should be selected to withstand any associated operating parameters. Alternatively, light source 32 can be mounted or affixed to outer surface 18 of housing member 12. In this regard, housing member 12 is made of a material that permits transmission of light energy from light source 32 through sidewall 16 of housing member 12.

Sensor system 14 can comprise a power source 34, non-volatile memory 36, a transmitter 38, and/or other operational systems 40 (such as optical systems, flow systems, camera systems, thermal systems, and the like) disposed within and/or in operational connection with a sensor housing 42. Power source 34 can be co-located with sensor housing 42 in the form of a battery or other portable power, such as solar, or may be connected via wires 44 to an external source 46. Transmitter 38 can be a wired or wireless transmitter outputting a sensor signal 26 to a device 28 via any known system, such as wire, data cable, Ethernet, BlueTooth®, radio frequency (RF), ZigBee, or any other communication system or protocol. It should be understood that sensor system 14 can be configured to record, save, broadcast, transmit, analyze, or otherwise process data during operation.

As illustrated in FIGS. 3 and 7, working fluid monitoring system 10 can further include a mesh or other obstruction member, such as a protrusion, 48 disposed within working fluid passage 25. Mesh 48 can be retained within a channel or other retention system 50 disposed within housing member 12. Mesh or obstruction member 48 is particularly configured to provide a screen for filtering the working fluid as it passes through working fluid passage 25 and/or to provide protection for sensor system 14 and/or light source 32 from damage caused by funnels, implements, or foreign objects inserted within fill pipe 102, 104 or fill port 106.

As illustrated in FIGS. 5 and 7, working fluid monitoring system 10 can include internal baffles 52 disposed within working fluid passage 25 to improve mixing and/or exposure of the working fluid to sensor system 14. Baffles 52 can further be used to slow the flowrate of working fluid there past to improve monitoring, detection, testing and/or validating.

In some embodiments, housing member 12 is sized to be coupled, disposed, and/or affixed to the adjacent structure of machinery 100. In some embodiments, as illustrated in FIG. 1, working fluid monitoring system 10 can comprise a cylindrical housing member 12 that is sized to permit insertion of working fluid monitoring system 10 into fill pipe 102. Housing member 12 can then engage fill pipe 102 such that outer surface 18 of housing member 12 contacts and maintains position of working fluid monitoring system 10 relative to fill pipe 102. This can occur via a press-fit, interference-fiction, or other frictional retention. It should be understood that an adhesive or other fastening system can be used between housing member 12 and fill pipe 102. In some embodiments where fill pipe 102 includes a bend 110, working fluid monitoring system 10 can define a sufficient axial length between ends 22 and 24 to permit working fluid monitoring system 10 to be captured in fill pipe 102 upstream of bend 110 to firmly retain working fluid monitoring to system 10 in position.

In some embodiments, as illustrated in FIG. 4, working fluid monitoring system 10 can comprise housing member 12 being shaped as a flange housing member 12′ that can be inserted or otherwise fastened between a flanged end 112 of flanged fill pipe 104 and engine 114. In this way, housing member 12′ can use existing structure and fastening system of flanged fill pipe 104 to mount and retain working fluid monitoring system 10 in operable communication with the working fluid. It should be understood that housing member 12′ can retain similar material and operational properties as housing member 12. This configuration would permit the reliable fastening of working fluid monitoring system 10 to machinery 100 and minimize the likelihood of damage to working fluid monitoring system 10 from a funnel or other implement. Moreover, working fluid monitoring system 10 can be fixedly retained in a known position that is not easily compromised.

In some embodiments, as illustrated in FIG. 7, working fluid monitoring system 10 can comprise housing member 12 being shaped as a threaded member for threadedly engaging existing threaded fill port 106 of machinery 100. Threaded fill port 106 is a conventional fill port having a conventional threaded cap member 116. According to the present embodiment, housing member 12 can comprise threaded ends 22′ and 24′. End 22′ can be threadedly engaged with threaded fill port 106 to fixedly retain working fluid monitoring system 10 to machinery 100. Likewise, conventional threaded cap member 116 can be threadedly engaged with end 24′ such that working fluid added to threaded fill port 106 first passes through working fluid passage 25 before entry into the sump of machinery 100.

In some embodiments, as illustrated in FIG. 6, working fluid monitoring system 10 can comprise housing member 12 being sized such that ends 22, 24 can receive existing tubing 108. That is, ends 22, 24 of housing member 12 can be sized such that an existing tubing 108 is cut or ends exposed and mounted to ends 22, 24 of housing member 12. It should be appreciated that ends 22, 24 of housing member 12 can be configured to receive the ends of tubing 108 therein or thereabout. One or more retention members 54, such as pipe clamps, can be fixed over the ends of tubing 108 and ends 22, 24 of housing member 12 to effect a reliable sealing connection according to conventional principles. In this way, working fluid monitoring system 10 can be placed or positioned within an existing tubing member to monitor, detect, test, and/or validate the working fluid during operation, rather than merely during filling. Accordingly, as described herein, the present teachings provide for ongoing and continuous monitoring of the working fluid.

In light of the foregoing, it should be appreciated that the present teachings provide a working fluid monitoring system that is particularly configured to be retroactively fitted to equipment and capable of in-line fluid monitoring. As discussed herein, the housing member design enables it to be installed in the fluid fill area, either in the fill pipe or directly to the fill port, or affixed in-line on an existing working fluid hose. The internal design of the housing member can be cylindrical and configured to encasing or otherwise affix one or more sensors, light sources, flowmeters, or other instrumentation. The design can include baffles to direct flow over the sensor and a screen to prevent debris from contacting the sensor internal instrumentation.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A working fluid monitoring system for monitoring a working fluid of a working fluid system of a piece of equipment, the working fluid monitoring system comprising:
a housing member having a working fluid passage for receiving the working fluid therein; and
a sensor system in operable communication with the working fluid within the working fluid passage of the housing member, the sensor system being configured to monitor in situ the working fluid within the working fluid passage and output a sensor signal.

2. The working fluid monitoring system of claim 1 wherein the housing member comprises a fill pipe of the piece of equipment, the fill pipe configured to receive the working fluid there through.

3. The working fluid monitoring system of claim 1 wherein the housing member is a hollow cylindrical member disposed within a fill pipe of the piece of equipment, the fill pipe and the housing member configured to receive the working fluid there through.

4. The working fluid monitoring system of claim 3 wherein the housing member engages the fill pipe of the piece of equipment to maintain a predetermined position within the fill pipe.

5. The working fluid monitoring system of claim 1 wherein the housing member is mounted within a fill port of the piece of equipment, the fill port configured to receive the working fluid there in.

6. The working fluid monitoring system of claim 5 wherein the housing member comprises a threaded section, the threaded section configured to threadedly engage corresponding threads of the fill port.

7. The working fluid monitoring system of claim 1 wherein the housing member comprises a flanged fitting on an end thereof configured to be coupled to a corresponding fitting on the piece of equipment.

8. The working fluid monitoring system of claim 1 wherein the housing member comprises a collared fitting on opposing ends thereof, the collared ends configured to be coupled with an existing pipe of the piece of equipment.

9. The working fluid monitoring system of claim 1 wherein the sensor system comprises a light source configured to output light within the housing member and upon the working fluid, wherein a sensor component of the sensor system is responsive to the working fluid when exposed to the light.

10. The working fluid monitoring system of claim 9 wherein the light source comprises an ultraviolet light source.

11. The working fluid monitoring system of claim 1 wherein the sensor system is an RGB light sensor system.

12. The working fluid monitoring system of claim 1 wherein the sensor system detects a fluorescence of the working fluid within the housing member.

13. The working fluid monitoring system of claim 1 wherein the sensor system is configured to monitor in situ a parameter of the working fluid.

14. The working fluid monitoring system of claim 1 wherein the sensor system is configured to detect a counterfeit working fluid.

15. The working fluid monitoring system of claim 1 wherein the sensor system is configured to monitor in situ a parameter of the working fluid chosen from the group consisting of suspended soot, dielectric, conductivity, capacitance, impedance, light transmission, acoustic resonance, vapor pressure, pressure, temperature, total acid number, total base number, pH, oxidation, viscosity, flow rate, flow volume, water, coolant, density, oil viscosity grade, VI, SSI (viscosity modifier shear stability), ferrous iron, fuel dilution, RGB color, florescence, phosphorescence, UV, infrared, X-ray, XRF, RF, refractive index, varnish, deposits, iron, lead, copper, aluminum, tin, vanadium, zinc, phosphorus, boron, calcium, magnesium, include zirconium, nitrogen, chlorine, silver, titanium, chromium, cobalt, nickel, molybdenum, yttrium, tungsten, gold, cerium, strontium, sulfur, particle count, and air concentration.

16. The working fluid monitoring system of claim 1 wherein the sensor system comprises a sensor component, wherein the sensor component is responsive to an energy, the housing member being configured to permit the energy to pass through the material of the housing member to the sensor component.

17. The working fluid monitoring system of claim 1 wherein the sensor system comprises a transmitter for transmitting the sensor signal.

18. The working fluid monitoring system of claim 17 wherein the transmitter is a wireless transmitter.

19. The working fluid monitoring system of claim 1 wherein the sensor system comprises a sensor component and a power source co-located with sensor component.

20. A fluid monitoring system for monitoring a fluid of a fluid system, the fluid monitoring system comprising:
a housing member having a fluid passage for receiving the fluid therein; and
a sensor system in operable communication with the fluid within the fluid passage of the housing member, the sensor system being configured to monitor in situ a parameter of the fluid within the fluid passage and output a sensor signal, the sensor system having:
a light source configured to output light within the housing member and upon the fluid,
a sensor component responsive to the fluid when exposed to the light, and
a transmitter for transmitting the sensor signal.
